(19) United States
(12) Patent Application Publication
(10) Pub. No.: US 2025/0177187 A1
(43) Pub. Date: Jun. 5, 2025

(54) FLUID COLLECTION ASSEMBLIES INCLUDING AT LEAST ONE LENGTH ADJUSTING FEATURE

(71) Applicant: PUREWICK CORPORATION, Covington, GA (US)

(72) Inventor: Camille Rose Newton, Bonsall, CA (US)

(21) Appl. No.: 19/038,774

(22) Filed: Jan. 28, 2025

Related U.S. Application Data
(63) Continuation of application No. 17/661,090, filed on Apr. 28, 2022, now Pat. No. 12,233,003.
(60) Provisional application No. 63/181,709, filed on Apr. 29, 2021.

(51) Int. Cl. A61F 5/453; A61F 5/44; A61M 1/00

(52) U.S. Cl. CPC A61F 5/453; A61F 5/4404; A61M 1/80

(57) ABSTRACT

Embodiments are directed towards fluid collection assemblies that include at least one length adjusting feature, fluid collection assemblies including the same, and methods of using the same. An example fluid collection assembly includes a fluid impermeable barrier. The fluid impermeable barrier includes a distal region, at least one distal end region spaced from the distal region, and at least one intermediate portion extending from the distal region to the distal region. The fluid impermeable barrier also defines at least one length adjusting feature (“LAF”) extending from the distal end region to the distal region. The LAF allows the length of the fluid collection assembly to change. The ability of the fluid collection assembly to change the length thereof allows a penis of a patient to be disposed in preferential locations within the fluid collection assembly.

### CROSS-REFERENCE TO RELATED APPLICATIONS

This application is a continuation application of U.S. patent application Ser. No. 17/661,090 filed on Apr. 28, 2022, which claims priority to U.S. Provisional Application No. 63/181,709 filed on Apr. 29, 2021, the disclosure of each of which is incorporated herein, in its entirety, by this references.

### BACKGROUND

A patient may have limited or impaired mobility such that typical urination processes are challenging or impossible. For example, the patient may have surgery or a disability that impairs mobility. In another example, the patient may have restricted travel conditions such as those experience by pilots, drivers, and workers in hazardous areas. Additionally, fluid collection from the patient may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections.

### SUMMARY

Embodiments are directed towards fluid collection assemblies that include at least one length adjusting feature, fluid collection systems including the same, and methods of using the same. In an embodiment, a fluid collection assembly is disclosed. The fluid collection assembly includes a distal region, at least one proximal end region spaced from the distal region, at least one intermediate portion extending from the at least one proximal end region to the distal region, and at least one length adjusting feature extending from the at least one proximal end region towards the distal region. The at least one length adjusting feature is configured to allow a length of the fluid collection assembly to change. The fluid collection assembly also includes a fluid impermeable barrier forming at least a portion of the distal region, the at least one proximal end region, and the at least one intermediate portion. The fluid impermeable barrier at least defining a chamber, an opening at the proximal end region, and a fluid outlet. The fluid collection assembly further includes at least one porous material disposed in the chamber.

In an embodiment, a fluid collection assembly is disclosed. The fluid collection assembly includes a fluid storage container configured to hold one or more bodily fluids. The fluid collection assembly also includes a fluid collection assembly. The fluid collection assembly includes a distal region, at least one proximal end region spaced from the distal region, at least one intermediate portion extending from the at least one proximal end region to the distal region, and at least one length adjusting feature extending from the at least one proximal end region towards the distal region. The at least one length adjusting feature is configured to allow a length of the fluid collection assembly to change. The fluid collection assembly also includes a fluid impermeable barrier forming at least a portion of the distal region, the at least one proximal end region, and the at least one intermediate portion. The fluid impermeable barrier at least defining a chamber, an opening at the proximal end region, and a fluid outlet. The fluid collection assembly further includes at least one porous material disposed in the chamber. The fluid collection system further includes a vacuum source fluidly coupled to one or more of the fluid storage container or the fluid collection assembly via the conduit. The vacuum source is configured to draw fluid from the fluid collection assembly via the conduit.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection assembly, according to an embodiment.

FIGS. 1B and 1C are cross-sectional schematics of the fluid collection assembly taken along planes 1B-1B and 1C-1C illustrated in FIG. 1A, respectively.

FIG. 1D is an isometric view of the fluid collection assembly shown in FIG. 1A during use, according to an embodiment.

FIG. 2A is a side elevational view of a fluid collection assembly that includes a length adjusting feature other than at least one slit, according to an embodiment.

FIG. 2B is a cross-sectional schematic of the fluid collection assembly taken along plane 2B-2B illustrated in FIG. 2A, according to an embodiment.

FIG. 3A is a cross-sectional schematic of a fluid collection assembly, according to an embodiment.

FIG. 3B is a cross-sectional schematic of the fluid collection assembly taken along plane 3B-3B shown in FIG. 3A.

FIG. 4A is an isometric view of a fluid collection assembly that includes a two intermediate portions, according to an embodiment.

FIG. 4B is an isometric view of the fluid collection assembly during use, according to an embodiment.

FIG. 5A is an isometric view of a fluid collection assembly, according to an embodiment.

FIG. 5B is a top plan view of the fluid collection assembly, according to an embodiment.

FIG. 5C is a top plan view of a fluid collection assembly that includes a plurality of intermediate portions that are not equidistantly spaced, according to an embodiment.

FIG. 6 is an isometric view of a fluid collection assembly that includes overlapping intermediate portions, according to an embodiment.

FIG. 7A is an isometric view of a fluid collection assembly, according to an embodiment.

FIG. 7B is a cross-sectional schematic of the fluid collection assembly being used with a penis, according to an embodiment.

FIG. 8 is an isometric view of a fluid collection assembly that includes a distal region that is not a sump, according to an embodiment.

FIG. 9 is a block diagram of a fluid collection system for fluid collection, according to an embodiment.

### DETAILED DESCRIPTION

Embodiments are directed towards fluid collection assemblies that include at least one length adjusting feature (“LAF”), fluid collection systems including the same, and methods of using the same. An example fluid collection assembly includes a fluid impermeable barrier. The fluid impermeable barrier includes a distal region, at least one proximal end region spaced from the distal region, and at least one intermediate portion extending from the distal region to the proximal end region. The fluid impermeable barrier also defines at least one LAF extending from the proximal end region towards the distal region. The LAF forms one or more lateral edges of the at least one intermediate portion. The LAF allows the length of the fluid collection assembly to change. The ability of the fluid collection assembly to change the length thereof allows a penis of a patient (i.e., an individual using the fluid collection assembly) to be disposed in preferential locations within the fluid collection assembly.

Condom male external catheters (“CMECs”) are devices that are configured to have a penis disposed therein and remove bodily fluids (e.g., urine) that are discharged from the penis. CMECs are generally configured to be used with any penis, regardless of size, to at least one of minimize inventory, allow the penis to become erect without compressing the erect penis, or to prevent the need to use different CMECs depending on whether the penis is flaccid or erect. As such, the CMECs may be configured to be used with penises that, when erect, exhibit an above average size (e.g., an erect penis exhibiting a length greater than 20 cm, measured from a tip of the penis, such as the urethral opening of the penis, to the location where the shaft meets the mons pubis) and, thus, the CMEC may exhibit a length that is greater than 8 inches. However, the CMECs may be used with buried penises, flaccid penises, and penises exhibiting average to below average sizes when erect. Due to the large length of the CMECs, CMECs may exhibit a large distance between the fluid outlet of the CMEC (which is typically at a distal end region of the CMEC) and the penis when the penis is buried, flaccid, or exhibits an average or below average size when erect. The large distance between the penis and the fluid outlet may allow the bodily fluids discharged from the penis to flow in the wrong direction (e.g., away from the fluid outlet) and allow for greater loss of vacuum when a vacuum is used to remove the bodily fluids from the CMECs. Bodily fluids flowing in the wrong direction and/or the loss of vacuum may prevent or inhibit the removal of the bodily fluids from the CMECs which may cause patient discomfort (i.e., the patient remains in contact with the bodily fluids), create unsanitary conditions, and increase the likelihood that the bodily fluids leak from the CMECs.

The fluid collection assemblies disclosed herein may resolve at least some of the above discussed issues of the CMECs. For example, the fluid collection assemblies may exhibit a maximum length that is sufficiently large that the fluid collection assemblies may be used with above average sized erect penises. The fluid collection assemblies may be positioned on a penis such that the urethral opening of the penis is adjacent or at least proximate to the fluid outlet and/or distal region of the fluid collection assemblies. The LAF allows the length of the fluid collection assembly to be effectively changed (e.g., reduced) such that the urethral opening of buried penises, flaccid penises, and erect penis exhibiting average or below average lengths may be positioned adjacent or at least proximate to the fluid outlet and/or the distal region. The LAF allows the portions of the fluid collection assembly to flare outwardly (e.g., increase a width thereof) or be folded to reduce the effective length of the fluid collection assembly. Thus, the LAF may reduce the distance from the urethral opening of the penis and the fluid outlet and/or distal region thereby inhibiting the bodily fluids flowing in the wrong direction and/or inhibiting loss of vacuum.

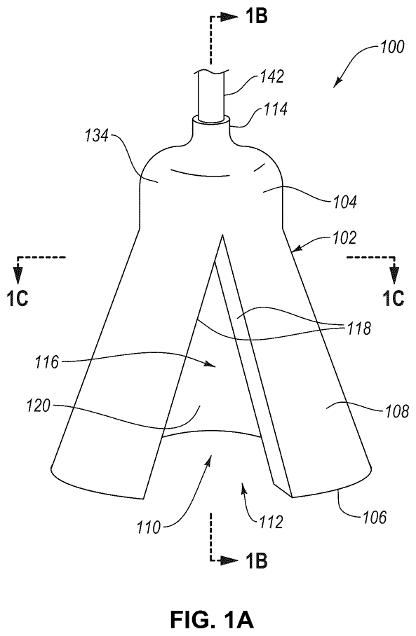

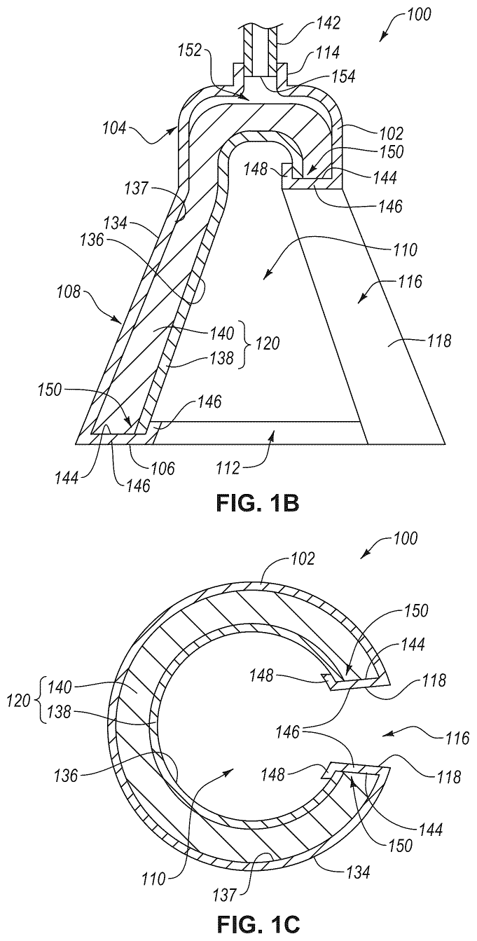

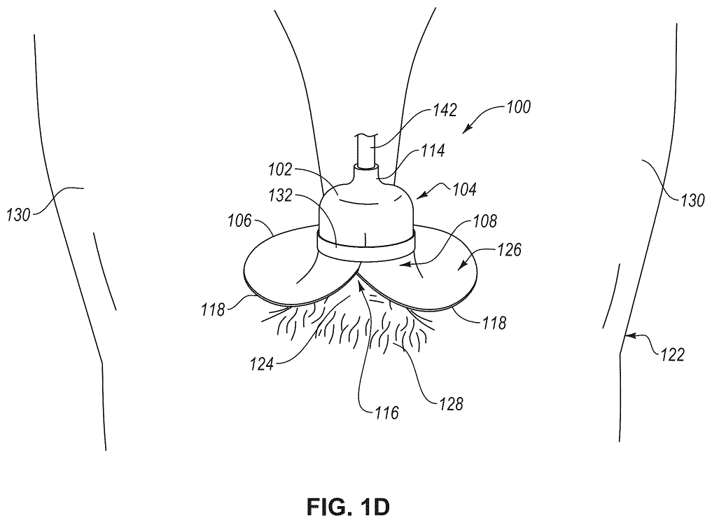

FIG. 1A is an isometric view of a fluid collection assembly 100, according to an embodiment. FIGS. 1B and 1C are cross-sectional schematics of the fluid collection assembly 100 taken along planes 1B-1B and 1C-IC illustrated in FIG. 1A, respectively, according to an embodiment. The fluid collection assembly 100 includes a distal region 104, a proximal end region 106, and an intermediate portion 108 extending from the distal region 104 to the proximal end region 106. The fluid collection assembly 100 includes a fluid impermeable barrier 102 that forms at least a portion of the distal region 104, the proximal end region 106, and the intermediate portion 108. The fluid impermeable barrier 102 also defines a chamber 110, an opening 112 at the proximal end region 106 that allows a penis 124 (partially shown in FIG. 1D) to be disposed in the chamber 110, a fluid outlet 114 at or near the distal region 104 that allows bodily fluids to be removed from the chamber 110, and at least one LAF extending from the proximal end region 106 to the distal region 104. In the illustrated embodiment, the LAF is a slit 116 that extends from the proximal end region 106 to the distal region 104. The LAF forms the lateral edges 118 of the intermediate portion 108. The fluid collection assembly 100 also includes at least one porous material 120 disposed in the chamber 110.

The distal region 104 is a portion of the fluid collection assembly 100 that is configured to have a urethral opening of the penis 124 disposed adjacent or at least proximate thereof. As such, the distal region 104 (e.g., the porous material 120 of the distal region 104) may receive bodily fluids that are discharged from the urethral opening preferentially and/or before the other portions of the fluid collection assembly 100. For example, a stream of bodily fluids that are omitted from the urethral opening may first contact the distal region 104 of the fluid collection assembly 100 though, it is noted, that the stream may contact other regions of the fluid collection assembly since the penis 124 may be flimsy when flaccid which may allow the bodily fluids to be omitted at a variety of angles even when the urethral opening is adjacent or proximate to the distal region 104.

The distal region 104 includes a portion of the fluid collection assembly 100 that is spaced from proximal end region 106 and the lateral edges 118 of the intermediate portion 108 (e.g., the LAF is not present in the distal region 104). For example, the proximal end region 106 and the lateral edges 118 represent locations through which the bodily fluids may leak from the chamber 110. As such, spacing the distal region 104 from the proximal end region 106 and the lateral edges 118 may minimize the likelihood that the bodily fluids leak from the fluid collection assembly 100.

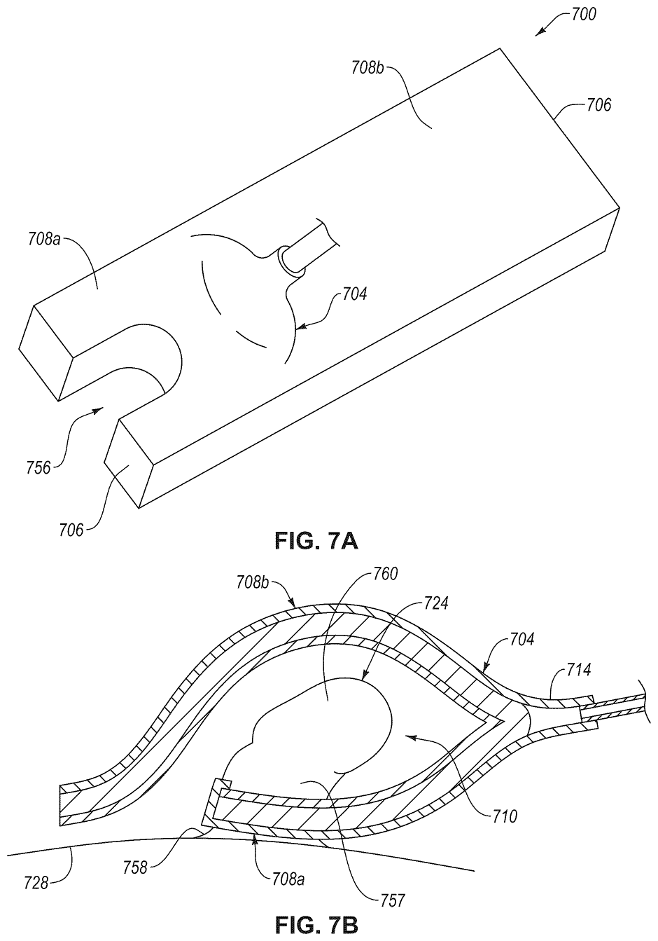

In an embodiment, as illustrated, the distal region 104 is spaced substantially equidistantly from the all portions of the proximal end region 106 which decreases the likelihood that the bodily fluids may preferentially leak from one portion of the proximal end region 106. In other words, the distal region 104 is located substantially in the middle of the fluid collection assembly 100. In an embodiment, as shown in FIGS. 7A and 7B, the distal region 104 may not be substantially equidistantly spaced from all the portions of the proximal end region 106. In other words, the distal region 104 is located off-center. However, in such an embodiment, the distal region 104 is still spaced from the proximal end region 106 and the lateral edges 118 to minimize leakage of the bodily fluids from the fluid collection assembly 100.

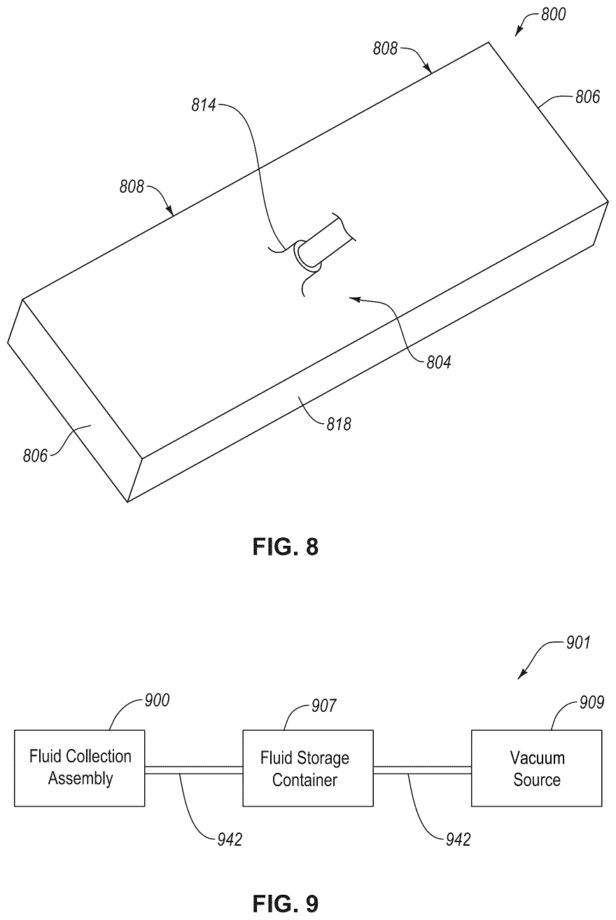

In an embodiment, as shown, the distal region 104 may form a sump. The distal region 104 forms a sump when the intermediate portion 108 is fully flared (e.g., the length and maximum width of the fluid collection assembly 100 cannot be smaller and bigger, respectively) and pressed against a flat surface, the portions of the distal region 104 in addition to the fluid outlet 114 may protrude upwardly from the intermediate portion 108. The sump provides a location for at least a portion of the glans (head) of the penis 124 to be disposed in. The sump may optionally provide a location for at least a portion of the shaft of the penis 124 to be disposed in. Providing a location for the glans of the penis to be positioned in may prevent inadvertently compressing the glans of the penis when the fluid collection assembly 100 is secured to the patient, as discussed in more detail below. Further, the sump may increase a distance from the urethral opening of the penis and the proximal end region 106 and the lateral edges 118 thereby decreasing the likelihood that the bodily fluids leak from the chamber 110. In an embodiment, as shown, the sump may exhibit a hollow generally cylindrical shape which generally corresponds to the shape of the penis. In an embodiment, the sump may exhibit a non-cylindrical shape, such as a generally spherical or conical shape. In an embodiment, illustrated in FIG. 8, the distal region 104 does not form a sump. In such an embodiment, the fluid outlet 114 may optionally protrude from the surface when the intermediate portion 108 is fully flared and pressed against the surface.

The fluid outlet 114 may be located on the distal region 104 since the urethral opening of the penis is positioned adjacent to or at least proximate to the distal region 104 thereby reducing the distance between the urethral opening and the fluid outlet 114. However, it is noted that the fluid outlet 114 may be spaced from the distal region 104. In an example, the fluid outlet 114 may be positioned on the intermediate portion 108 at a location that is proximate to (e.g., within about 8 cm, within about 6 cm, within about 5 cm, within about 4 cm, within about 3 cm, within about 2cm, within about 1 cm, within about 0.5 cm, within 0.25 cm of) the distal region 104. In an example, the fluid outlet 114 may be positioned at or near an expected gravimetric low point of the chamber 110 since at least some of the bodily fluids, once received by the porous material 120, may generally flow in the direction of gravity and the distal region 104 may not be located at the gravimetric low point of the chamber 110.

As previously discussed, the fluid collection assembly 100 includes an LAF. In the illustrated embodiment, the LAF includes a slit 116 that extends from the proximal end region 106 towards (e.g., to) the distal region 104. The slit 116 is configured to control the length of the fluid collection assembly 100. In particular, the slit 116 allows at least a portion of the intermediate portion 108 to be flared (e.g., a width thereof increased) and/or folded back onto the rest of the fluid collection assembly 100. Flaring the intermediate portion 108 or folding the intermediate portion 108 back onto the rest of the fluid collection assembly 100 decreases the effective length of the fluid collection assembly 100. As used herein, the effective length refers to the maximum distance the fluid collection assembly 100 may extend from a base of a penis 124 when at least a portion of the fluid collection assembly 100 is adjacent to the base of the penis 124.

FIG. 1D is an isometric view of the fluid collection assembly 100 during use, according to an embodiment. FIG. 1D illustrates how the LAF (e.g., slit 116) allows the length of the fluid collection assembly 100 to change. FIG. 1D illustrates a patient 122 having a penis 124 (partially obscured) that exhibits a length that is smaller than a maximum length of the fluid collection assembly 100 (e.g., a length of the fluid collection assembly 100 when no portion of the intermediate portion 108 is flared or folded). It is desirable for the urethral opening of the penis 124 to be disposed in, adjacent, or proximate to the distal region 104 for reasons previously discussed. The LAF allows the length of the fluid collection assembly 100 to be decreased such that the urethral opening of the penis 124 is in, adjacent, or proximate to the distal region 104.

In an embodiment, as shown, the length of the fluid collection assembly 100 may be changed (e.g., decreased) by flaring at least a portion of the intermediate portion 108. Flaring at least a portion of the intermediate portion 108 refers to increasing the width of at least a portion of the intermediate portion 108. The portions of the intermediate portion 108 that flare are referred to as the flared portions 126. For example, the width of the intermediate portion 108 may be increased until flared portion 126 is generally flat or exhibits a shape that generally corresponds to the shape of the region about the penis 124 (e.g., the mons pubis 128, the thighs 130, the testicles (not shown), or other anatomical feature about the penis 124). The flared portion 126 may be formed by moving corresponding portions of the lateral edges 118 (i.e., portions of the lateral edges 118 that are adjacent to each other when the slit 116 is closed) of the intermediate portion 108 away from each other. That is, the flared portion 126 may be formed by increasing a distance between corresponding portions of the lateral edges 118. Increasing the width of the flared portion 126 decreases the length of the fluid collection assembly 100.

In an embodiment, the length of the fluid collection assembly 100 may be changed by folding at least a portion of the intermediate portion 108 back onto itself and/or onto the distal region 104. In such an embodiment, folding the intermediate portion 108 may be formed, at least initially, by flaring at least a portion of the intermediate portion 108 to form a flared portion 126. After forming the flared portion 126, corresponding portions of the lateral edges 118 are brought back together (e.g., a distance between corresponding portions of the lateral edges 118 are decreased) such that an interior of at least a portion of the intermediate portion 108 faces outwardly. For example, folding the intermediate portion 108 may cause a portion of the porous material 120 to be exposed. In other words, the fluid impermeable barrier 102 of the portions of the intermediate portion 108 that were initially flared are positioned proximate to the fluid impermeable barrier 102 of the portions of the fluid collection assembly 100 that were not flared.

The change in the length of the fluid collection assembly 100 is controlled by either flaring or folding all, some, or none of the intermediate portion 108. In particular, increasing the percentage of the intermediate portion 108 that is flared or folded decreases the length of the fluid collection assembly 100. For example, flaring or folding none of the intermediate portion 108 maintains the maximum length of the fluid collection assembly 100, flaring or folding all of the intermediate portion 108 minimizes the length of the collection assembly 100, and flaring or folding only a portion of the intermediate portion 108 causes the fluid collection assembly 100 to exhibit a length that is between the maximum and minimum length. When only a portion of the intermediate portion 108 is flared or folded, greater than 0% to about 20%, about 10% to about 30%, about 20% to about 40%, about 30% to about 50%, about 40% to about 60%, about 50% to about 70%, about 60% to about 80%, about 70% to about 90%, or about 80% to about 99% of a length of the intermediate portion 108 may be flared or folded. Whether all, none, or only some of the intermediate portion 108 is flared or folded may be selected based on the size of the penis 124 that the fluid collection assembly 100 is used with so that the length of the fluid collection assembly 100 allows the urethral opening of the penis 124 to be positioned within, adjacent, or proximate to the distal region 104.

In an embodiment, the fluid collection assembly 100 may exhibit a maximum length that is greater than about 14 cm, such as about 14 cm to about 16 cm when configured to be used with average or below average sized penises only or greater than 16 cm (e.g., about 16 cm to about 20 cm or about 18 cm to about 22 cm) when configured to be used with substantially all penises. The maximum length is the maximum distance from the distal region 104 to the proximal end region 106. The fluid collection assembly 100 may exhibit a final length after at least a portion of the intermediate portion 108 is flared or folded. The final length is less than the maximum length. For example, the final length may be 0 (the distal region 104 does not include a sump) to about 2 cm, about 1 cm to about 3 cm, about 2 cm to about 4 cm, about 3 cm to about 5 cm, about 4 cm to about 6 cm, about 5 cm to about 7 cm, about 6 cm to about 8 cm, about 7 cm to about 9 cm, about 10 cm to about 12.5 cm, about 12 cm to about 15 cm, or about 14 cm to about 20 cm. The final length may be the minimum length of the fluid collection assembly 100 or a length between the maximum length and the minimum length.

In an embodiment, as previously discussed, none or only a portion of the intermediate portion 108 may be flared or folded. In such an embodiment, the fluid collection assembly 100 may include at least one retainer 132. The retainer 132 may be configured to prevent unflared or folded portions of the intermediate portion 108 from flaring or folded (e.g., prevent a distance between corresponding portions of the lateral edges 118 that do not form part of the flared portion 126 from increasing). For example, the retainer 132 may maintain the lateral edges 118 adjacent (e.g., abutting) to each other or overlap portions of the intermediate portion 108, either of which may minimize leakage through the slit 116. The retainer 132 may be positioned on (e.g., attached to, disposed on, fitted around) an exterior surface 134 of the fluid impermeable barrier 102 thereby preventing or at least inhibiting the distance between the corresponding portions lateral edges 118 adjacent to retainer 132 or between the retainer 132 and the distal region 104 from increasing. As such, the retainer 132 may inhibit a length of the fluid collection assembly 100 from changing after positioning the retainer 132 on the exterior surface 134. In an example, the retainer 132 may maintain the lateral edges 118 adjacent (e.g., abutting) to each other.

In an example, the retainer 132 may cause the unflared or unfolded portions of the intermediate portions 108 to overlap. Overlapping the unflared or unfolded portions of the intermediate portions 108 may cause the fluid collection assembly 100 to clasp a shaft of the penis 124 thereby facilitating attachment of the fluid collection assembly 100 to the patient 122. Overlapping the unflared or unfolded portions of the intermediate portions 108 may also decrease the volume of the chamber 110, thereby increasing the likelihood that the bodily fluids discharged into the chamber 110 are received into the porous material 110 instead of leaking. Other structures that may be used to clasp against the shaft of the penis 134, thereby facilitating attachment of the fluid collection assembly 100 to the patient 122 are disclosed in U.S. Provisional Patent Application No. 63/133,892 filed on Jan. 5, 2021, the disclosure of which is incorporated herein, in its entirety, by this reference.

The retainer 132 may include any device that may prevent the distance between corresponding portions of the lateral edges 118 adjacent to the retainer 132 and/or between the retainer 132 and the distal region 104 from increasing. In an example, the retainer 132 may include tape that is disposed across the slit 116. In an example, the retainer 132 may include a sleeve (e.g., ring) that fits around the exterior surface 134. The sleeve may not be adhesively attached to the exterior surface 134 of the fluid impermeable barrier 102 and, instead, may rely on friction to maintain the sleeve on the exterior surface 134. In an example, the retainer 132 may include a Velcro strap that is attached to and/or extends around the exterior surface 134. In an example, the retainer 132 may include an elastic band, rope or other string, or any other suitable device.

The intermediate portion 108 is defined by at least the distal region 104, the proximal end region 106, and the lateral edges 118 of the intermediate portion 108. The shape of the intermediate portion 108 may depend whether the intermediate portion 108 is unflared or flared. For example, the intermediate portion 108 may exhibit a generally cylindrical shape when the intermediate portion 108 is unflared, a generally truncated cylindrical shape when the intermediate portion 108 is flared, and a generally flat shape when the intermediate portion 108 is completely flared. The intermediate portion 108 may exhibit other shapes, such as a generally rectangular shape, a generally semi-cylindrical shape, at least a portion of a generally parallelepiped shape, or any other suitable shape.

In an embodiment, the intermediate portion 108 is configured to attach the fluid collection assembly 100 to the patient 122. In such an embodiment, at least a portion of the intermediate portion 108 includes an adhesive (e.g., hydrogel) on an interior surface 137 of the fluid impermeable barrier 104 or the interior surface 136 of the porous material 120. The adhesive may be configured to attach the intermediate portion 108 to the shaft of the penis and/or the region about the penis 124 thereby securing the fluid collection assembly 100 to the patient. In an example, the portions of the fluid impermeable barrier 102 that form a portion of the interior surface 137 (e.g., the inwardly extending portion 146 and/or the chamber portion 148) may include the adhesive. In an example, at least a portion of the porous material 120 that forms the interior surface 136 includes the adhesive. In an example, only portions of the interior surface 136 adjacent or proximate to the proximal end region 106 and the lateral edges 118 include the adhesive. In such an example, the adhesive may be configured to form a seal that prevents the bodily fluids from flow out of the chamber 110.

Referring back to FIGS. 1A-1C, as previously discussed, the fluid collection assembly 100 includes a fluid impermeable barrier 102. The fluid impermeable barrier 102 temporarily stores the bodily fluids discharged from a penis in the chamber 110. The fluid impermeable barrier 102 stores the bodily fluids in the chamber 110. The fluid impermeable barrier 102 may be formed of any suitable fluid impermeable material(s), such as a fluid impermeable polymer (e.g., silicone, polypropylene, polyethylene, polyethylene terephthalate, neoprene, a polycarbonate, etc.), a metal film, natural rubber, another suitable material, or combinations thereof. As such, the fluid impermeable barrier 102 substantially prevents the bodily fluids from passing through the fluid impermeable barrier 102. In an example, the fluid impermeable barrier 102 may be air permeable and fluid impermeable. In such an example, the fluid impermeable barrier 102 may be formed of a hydrophobic material that defines a plurality of pores. At least one or more portions of at least an exterior surface 134 of the fluid impermeable barrier 102 may be formed from a soft and/or smooth material, thereby reducing chaffing.

The fluid impermeable barrier 102 at least partially defines the chamber 110. For example, the interior surface 137 of the fluid impermeable barrier 102 at least partially defines the perimeter of the chamber 110. The chamber 110 may at least temporarily retain fluids therein. In an example, portions of the chamber 110 may be substantially empty due to the varying sizes and rigidity of the male penis. However, in some examples, the outermost regions of the chamber 110 may include porous material 120 (e.g., one or more of the fluid permeable membrane 138 and fluid permeable support 140). For example, the porous material 120 may be bonded to at least a portion of the interior surface 137 of the fluid impermeable barrier 102. The porous material 120 may be positioned (e.g., at the distal end of the chamber 110) to blunt a stream of urine from the male urethra thereby limiting splashing and/or to direct the bodily fluids to a selected region of the chamber 110. Since the chamber 110 is substantially empty, the fluids are likely to pool at a gravimetrically low point of the chamber 110. The gravimetrically low point of the chamber 110 may be at an intersection of the skin of a patient and the fluid collection assembly 100, the distal region 104, or another suitable location depending on the orientation of the patient. It is noted that the varying length of the fluid collection assembly 100 may allow the penis to substantially completely occupy any portions of the chamber 110 that are not occupied by the porous material 120 or that forms the reservoir 152. Substantially completely occupying such portions of the chamber 110 may position the porous material 120 adjacent to the urethral opening, thereby causing the porous material 120 to receive substantially all of the bodily fluids discharged from the urethral opening and prevent the bodily fluids remaining in the unoccupied portions of the chamber 110.

The fluid impermeable barrier 102 may also define an opening 112 extending through the fluid impermeable barrier 102 that is configured to have the penis 124 positioned therethrough. The opening 112 may be defined by the portions of the fluid impermeable barrier 102 that form the proximal end region 106. For example, the opening 112 is formed in an extends through the fluid impermeable barrier 102, from the exterior surface 134 to the interior surface 137, thereby enabling the penis 124 and bodily fluids (e.g., when the penis 124 is buried) to enter the chamber 110. The opening 112 may also be formed in and extend through the porous material 120.

In some examples, the fluid impermeable barrier 102 may define the fluid outlet 114 that is sized to receive the conduit 142. The at least one conduit 142 may be disposed in the chamber 110 via the fluid outlet 114. The fluid outlet 114 may be sized and shaped to form an at least substantially fluid tight seal against the conduit 142 or the at least one tube thereby substantially preventing the bodily fluids from escaping the chamber 110.

The porous material 120 may include one or more of the fluid permeable membrane 138 or the fluid permeable support 140. One or more of the fluid permeable membrane 138 or the fluid permeable support 140 may be disposed between the fluid impermeable barrier 102 and a penis inserted into the chamber 110. The fluid permeable membrane 138 may be positioned between the fluid impermeable barrier 102 and a penis inserted into the chamber 110, such as between the fluid permeable support 140 and penis of a patient as shown. The fluid permeable support 140 may be positioned between the fluid permeable membrane 138 and the fluid impermeable barrier 102. The interior surface 137, optionally including the end of the chamber 110 substantially opposite the opening 112, may be covered with one or both the fluid permeable membrane 138 or the fluid permeable support 140. The fluid permeable support 140 or the fluid permeable membrane 138 may be affixed (e.g., adhered) to the fluid impermeable barrier 102. The fluid permeable support 140 or the fluid permeable membrane 138 may be affixed to each other.

The fluid collection assembly 100 includes assembly porous material 120 disposed in the chamber 110. The assembly porous material 120 may cover at least a portion (e.g., all) of the opening 112. The assembly porous material 120 may include a fluid permeable membrane 138 and a fluid permeable support 140. The assembly porous material 120 is exposed to the environment outside of the chamber 110 through the opening 112. In an embodiment, the assembly porous material 120 may be configured to wick any bodily fluids away from the opening 112, thereby preventing the bodily fluids from escaping the chamber 110. The permeable properties referred to herein may be wicking, capillary action, diffusion, or other similar properties or processes, and are referred to herein as “permeable” and/or “wicking.” Such “wicking” and/or “permeable” properties may not include absorption of the bodily fluids into at least a portion of the wicking material, such as not include adsorption of the bodily fluids into the fluid permeable support 140. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the wicking material (e.g., absorbency), such as less than about 30 wt % of the dry weight of the wicking material, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the wicking material. The porous material may also wick the bodily fluids generally towards an interior of the chamber 110, as discussed in more detail below. In an embodiment, the assembly porous material 120 may include at least one absorbent or adsorbent material.

The fluid permeable membrane 138 may be composed to wick the bodily fluids away from the opening 112, thereby preventing the bodily fluids from escaping the chamber 110. The fluid permeable membrane 138 may include any material that may wick the bodily fluids. For example, the fluid permeable membrane 138 may include fabric, such as a gauze (e.g., a silk, linen, or cotton gauze), another soft fabric, or another smooth fabric. Forming the fluid permeable membrane 138 from gauze, paper, soft fabric, and/or smooth fabric may reduce chaffing caused by the fluid collection assembly 100. In an embodiment, the fluid permeable membrane 138 may become quickly saturated with the bodily fluids after the porous material 120 received the bodily fluids from the penis. The fluid permeable membrane 138 may become substantially impermeable to gases when the fluid permeable membrane 138 becomes saturated with the bodily fluids thereby preventing or at least minimizing loss of a vacuum through the fluid permeable membrane 138. Preventing or minimizing the loss of the vacuum through the fluid permeable membrane 138 may increase the quantity of and rate at which the bodily fluids are removed from the chamber 110.

The fluid collection assembly 100 may include the fluid permeable support 140 disposed in the chamber 110. The fluid permeable support 140 is configured to support the fluid permeable membrane 138 since the fluid permeable membrane 138 may be formed from a relatively foldable, flimsy, or otherwise easily deformable material. For example, the fluid permeable support 140 may be positioned such that the fluid permeable membrane 138 is disposed between the fluid permeable support 140 and the fluid impermeable barrier 102. As such, the fluid permeable support 140 may support and maintain the position of the fluid permeable membrane 138. The fluid permeable support 140 may include any material that may wick or be permeable to the bodily fluids, such as any of the fluid permeable membrane materials disclosed herein above. For example, the fluid permeable membrane material(s) may be utilized in a more dense or rigid form than in the fluid permeable membrane 138 when used as the fluid permeable support 140. The fluid permeable support 140 may be formed from any fluid permeable material that is less deformable than the fluid permeable membrane 138. For example, the fluid permeable support 140 may include a porous polymer (e.g., nylon, polyester, polyurethane, polyethylene, polypropylene, etc.) structure or an open cell foam, such as spun nylon fiber. In some examples, the fluid permeable support 140 may be formed from a natural material, such as cotton, wool, silk, or combinations thereof. In an example, the fluid permeable support 140 may include a nonwoven material or a woven material (e.g., spun nylon fibers). In some examples, the fluid permeable support 140 may be formed from fabric, felt, gauze, or combinations thereof.

In an embodiment, the fluid permeable support 140 may be hydrophobic. The fluid permeable support 140 may be hydrophobic when the fluid permeable support 140 exhibits a contact angle with water (a major constituent of bodily fluids) that is greater than about 90°, such as in ranges of about 90° to about 120°, about 105° to about 135°, about 120° to about 150°, about 135° to about 175°, or about 150° to about 180°. The hydrophobicity of the fluid permeable support 140 may limit absorption, adsorption, and solubility of the bodily fluids in the fluid permeable support 140 thereby decreasing the amount of bodily fluids held in the assembly porous material 120. In an embodiment, the fluid permeable membrane 138 is hydrophobic or hydrophilic. In an embodiment, the fluid permeable support 140 is more hydrophobic (e.g., exhibits a larger contact angle with water) than the fluid permeable membrane 138. The lower hydrophobicity of the fluid permeable membrane 138 may help the assembly porous material 120 receive the bodily fluids from the urethral opening while the hydrophobicity of the fluid permeable support 140 limits the bodily fluids that are retained in the assembly porous material 120.

In some examples, the fluid permeable membrane 138 may be optional. For example, the porous material 120 may include only the fluid permeable support 140. In some examples, the fluid permeable support 140 may be optionally omitted from the fluid collection assembly 100. For example, the porous material 120 may only include the fluid permeable membrane 138. In some examples, the porous material 120 may include one or more additional layers in addition to or instead of at least one of the fluid permeable membrane 138 or the fluid permeable support 140.

In an embodiment, at least a portion of the porous material 120 at or near the proximal end region 106 may exhibit a tube-like structure (not shown) extending circumferentially about the proximal end region between the LAF. The tube-like structure of the porous material may include the fluid permeable membrane 138 rolled up into a generally curved cylindrical shape that may or may not define a channel. The fluid permeable support 140 may surround the fluid permeable membrane 138. Examples of a porous material 120 that exhibits a tube-like structure is disclosed in U.S. Pat. No. 10,376,406 filed on Jul. 27, 2016, the disclosure of which is disclosed herein, in its entirety, by this reference.

In an embodiment, the porous material 120 may be adjacent to substantially all of the interior surface 137 of the fluid impermeable barrier 102 such that there are substantially no gaps between the porous material 120 and the fluid impermeable barrier 102 ignoring the inherent porosity of the porous material 120. In an embodiment, the porous material 120 may not be adjacent to one or more portions of the interior surface 137 of the fluid impermeable barrier 102. In such an embodiment, the chamber 110 includes at least one reservoir 152 that is a substantially unoccupied portion of the chamber 110 that is between the fluid impermeable barrier 102 and the porous material 120. The bodily fluids that are in the chamber 110 may flow through the porous material 120 to the reservoir 152. The reservoir 152 may retain the bodily fluids therein until the bodily fluids are removed from the chamber 110. The reservoir 152 is depicted in the distal region 104 since the fluid outlet 114 is at or near the distal region 104. However, the reservoir 152 may be located in any portion of the chamber 110 between the fluid impermeable barrier 102 and the porous material 120, such as the proximal end region 106. The reservoir 152 may be located adjacent or proximate to the fluid outlet 114 or a gravimetrically low point of the fluid collection assembly 100 when the fluid collection assembly 100 is worn.

The fluid impermeable barrier 102 and the porous material 120 may be configured to prevent leaks from the chamber 110. In an example, the fluid impermeable barrier 102 may be configured such that the fluid impermeable barrier 102 that defines at least a portion of the lateral edges 118 contact each other when the when the slit 116 is closed (e.g., the lateral edges 118 contact each other or a portion of the intermediate portion 108 overlaps another portion of the intermediate portion 108). The fluid impermeable barrier 102 contacting itself prevents the formation of gaps through which the bodily fluids may leak and vacuum may be lost. In an example, as previously discussed, the fluid permeable membrane 138 may be configured to be substantially impermeable to gas when the fluid permeable membrane 138 becomes saturated with the bodily fluids. The saturated fluid permeable membrane 138 decreases vacuum loss which pulls the bodily fluids towards the fluid outlet 114, inhibits movement of the bodily fluids towards the slit 116 and the opening 112, and increases the quantity and rate at which the bodily fluids are removed from the chamber 110.

In an example, the fluid impermeable barrier 102 at least partially covers (e.g., extends around at least a portion of) one or more edges 144 of the porous material 120. In such an example, the fluid impermeable barrier 102 may include at least one inwardly extending portion 146 extending inwardly from a portion of the fluid impermeable barrier 102 that extends generally parallel to the porous material 120. The fluid impermeable barrier 102 may optionally include includes at least one chamber portion 148 extending from the inwardly extending portion 146 into the chamber 110. The inwardly extending portion 146 and/or the chamber portion 148 of the fluid impermeable barrier 102 form at least one of at least a portion of the proximal end region 106 (e.g., define a portion of the opening 112) or at least a portion of the lateral edges 118. The inwardly extending portion may prevent or at least inhibit bodily fluids from flowing out of the porous material 120 through the edges 144 thereof. The inwardly extending portion may also prevent or at least inhibit the vacuum being lost through the edges 144 of the porous material 120. The inwardly extending portion 146 and the chamber portion 148 of the fluid impermeable barrier 102 may also form a channel 150 that may store bodily fluids that reach the proximal end region 106 or the lateral edges 118. The bodily fluids stored in the channel 150 may be pulled from the channel 150 as bodily fluids are removed from the chamber 110 due to hydrogen bonding. The inwardly extending portion 146 and/or the chamber portion 148 may also increase contact between the fluid impermeable barrier 102 when the slit 116 is closed and/or a portion of the intermediate portion 108 overlaps another portion of the intermediate portion 108. The increased contact between the fluid impermeable barrier 102 reduces the likelihood that the bodily fluids leak through the slit 116.

The conduit 142 may be used to remove the bodily fluids from the chamber 110. The conduit 142 (e.g., a tube) includes an inlet 154 and an outlet (not shown) positioned downstream from the inlet 154. The outlet may be operably coupled to a vacuum source, such as a vacuum pump for withdrawing fluid from the chamber 110 through the conduit 142. For example, the conduit 142 may extend into the fluid impermeable barrier 102 from to the fluid outlet 114 to a point proximate to the reservoir 152 therein such that the inlet 154 is in fluid communication with the reservoir 152. The conduit 142 fluidly couples the chamber 110 with the fluid storage container (not shown) or the vacuum source (not shown).

The conduit 142 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some examples, the conduit 142 may include silicon or latex. In some examples, the conduit 142 may include one or more portions that are resilient, such as to by having one or more of a diameter or wall thickness that allows the conduit to be flexible.

As described in more detail below, the conduit 142 is configured to be coupled to, and at least partially extend between, one or more of the fluid storage container (not shown) and the vacuum source (not shown). In an example, the conduit 142 is configured to be directly connected to the vacuum source (not shown). In such an example, the conduit 142 may extend from the fluid impermeable barrier 102 by at least one foot, at least two feet, at least three feet, at least six feet, or at least eight feet. In another example, the conduit 142 is configured to be indirectly connected to at least one of the fluid storage container (not shown) and the vacuum source (not shown). In some examples, the conduit is secured to a patient's skin with a catheter securement device, such as a STATLOCK® catheter securement device available from C. R. Bard, Inc., including but not limited to those disclosed in U.S. Patent Nos. 6,117, 163; 6, 123,398; and 8,211,063, the disclosures of which are all incorporated herein by reference in their entirety.

The inlet 154 and the outlet are configured to fluidly couple (e.g., directly or indirectly) the vacuum source (not shown) to the chamber 110 (e.g., the reservoir 152). As the vacuum source (FIG. 9) applies a vacuum/suction in the conduit 142, the bodily fluids in the chamber 110 (e.g., at the distal region 104, such as in the reservoir 152) may be drawn into the inlet 154 and out of the fluid collection assembly 100 via the conduit 142. In some examples, the conduit may be frosted or opaque (e.g., black) to obscure visibility of the bodily fluids therein.

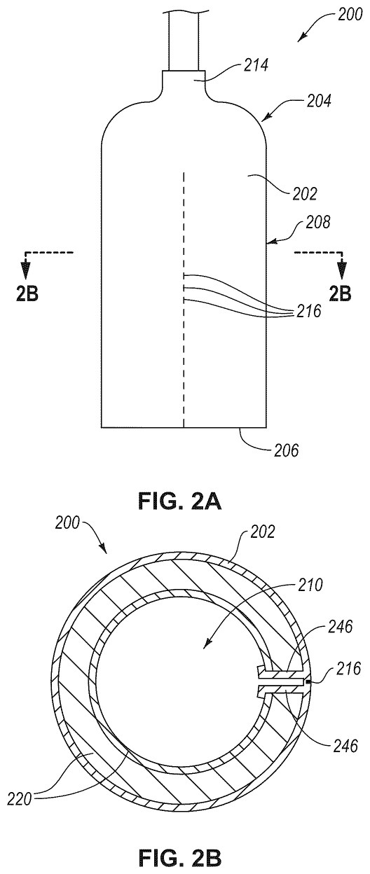

The slit 116 illustrated in FIGS. 1A-1D is merely one example of the LAF that may be included in any of the fluid collection assemblies disclosed herein. FIG. 2A is a side elevational view of a fluid collection assembly 200 that includes a LAF other than at least one slit, according to an embodiment. FIG. 2B is a cross-sectional schematic of the fluid collection assembly 200 taken along plane 2B-2B illustrated in FIG. 2A, according to an embodiment. Except as otherwise disclosed herein, the fluid collection assembly 200 is the same of substantially similar to any of the fluid collection assemblies disclosed herein. For example, the fluid collection assembly 200 includes a distal region 204, a proximal end region 206, at least one intermediate portion 208 extending from the distal region 204 to the proximal end region 206, and at least one LAF. The fluid collection assembly also includes a fluid impermeable barrier 202 defining a chamber 210 and at least one porous material 220 disposed in the chamber 210.

The LAF of the fluid collection assembly 200 includes a plurality of perforations 216 that extend through at least a portion of the fluid impermeable barrier 202. The perforations 216 may be arranged in a path, such as a substantially straight path. The plurality of perforations 216 may extend from the proximal end region 206 to the distal region 204. The perforations 216 weaken the fluid impermeable barrier 202 such that the fluid impermeable barrier 202 is configured to tear along the path. Tearing the fluid impermeable barrier 202 along the path formed by the perforations 216 forms a slit that functions substantially similar to the slit 116 illustrated in FIGS. 1A-1D.

The slit formed using the perforations 216 does not need to extend the whole length of the path formed by the perforations 216. Instead, the slit formed using the perforations 216 may only extend a portion of the length of the path depending on the size of the penis that the fluid collection assembly 200 is used with. For example, the slit formed using the perforations 216 may only extend about greater than 0% to about 20%, about 10% to about 30%, about 20% to about 40%, about 30% to about 50%, about 40% to about 60%, about 50% to about 70%, about 60% to about 80%, about 70% to about 90%, or about 80% to about 100% the length of the path formed by the perforations 216. The percentage of the length of the path that the slit extends may be selected based on the length of the path, the maximum length of the fluid collection assembly 100, the final length of the fluid collection assembly 200, and the length of the penis. Only tearing the a portion of the path formed by the protrusion 216 allows the slit to exhibit a length that was specifically selected for a specific sized penis. In other words, the length of the slit formed from the perforations may be selected to only be as large as necessary. The selected length of the slit formed from the perforations 216 may exhibit a length that is less than the slit 116 illustrated in FIGS. 1A-1D thereby decreasing the likelihood that the bodily fluids leak from the chamber 210.

Referring to FIG. 2B, the perforations 216 may only extend a portion of a distance through the fluid impermeable barrier 202. Only extending the perforations 216 through a portion of the fluid impermeable barrier 202 may prevent bodily fluids leaking through perforations 216 that were not torn. When the fluid impermeable barrier 202 includes two inwardly extending portions 246, the perforations 216 may be positioned between the two inwardly extending portions 246.

The LAF may include features other than a slit or a plurality of perforations. In an example, the LAF may include a portion of the fluid collection assembly that is weakened, such as by selectively thinning a portion of one or more components of the fluid collection assembly or forming a portion of one or more components of the fluid collection assembly from a weaker, more easily tear-able material. The weakened portions may facilitate tearing of a portion of the fluid collection assembly similar to the plurality of perforations. In an example, the LAF may include a flexible material that extends across a recess that extends through the fluid impermeable barrier and porous material. The flexible material and the recess allows the intermediate portion of the fluid collection assembly to flare but the flexible material may prevent or at least inhibit flow of bodily fluids through the recess. In an example, the LAF may include a combination of any of the LAFs disclosed herein, such as a slit and a plurality of protrusions extending from the slit towards the distal region.

Referring back to FIG. 1B, the inlet 154 of the conduit 142 is positioned to be within the fluid inlet 114, within the fluid reservoir 152, or adjacent to the porous material 120 that is in or adjacent to the distal region 104. The inlet 154 of the conduit 142 may facilitate removal of the bodily fluids from the chamber 110 when the distal region 104 is or near the gravimetric low point of the chamber 110. For example, the bodily fluids received by the porous material 120 may predominately flow in the direction of gravity towards the distal region 104 which allows the conduit 142 to remove the bodily fluids. The conduit 142 may still remove bodily fluids when the distal region 104 is not at or near the gravimetric low point of the chamber 110. For example, the conduit 142 may still receive some of the bodily fluids and the bodily fluids received by the conduit 142 may pull additional quantities of the bodily fluids into the conduit 142 due to hydrogen bonding. However, the conduit 142 may be unable to remove bodily fluids from portions of the chamber 110 spaced from the distal region 104 when the porous material 120 is not saturated with bodily fluids and when the distal region 104 is not at or near the gravimetric low point of the chamber 110.

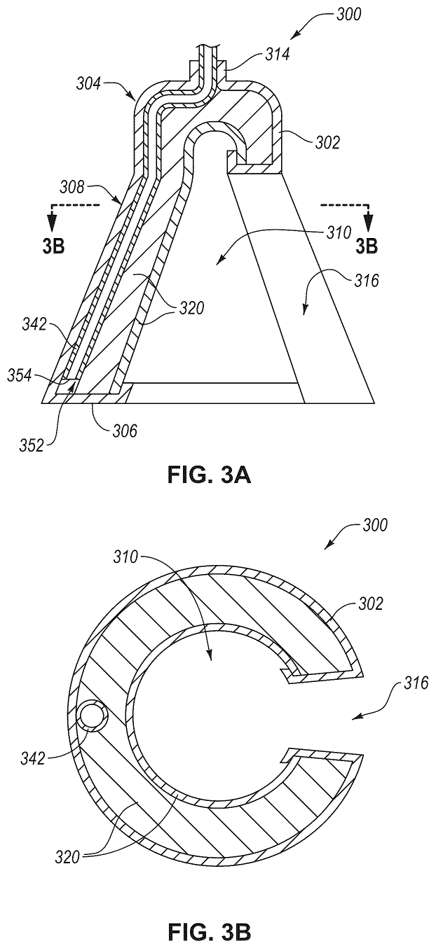

As such, the fluid collection assemblies disclosed herein may include positioning the inlet of the conduit at or near the expected gravimetric low point of the chamber even when the expected gravimetric low point of the chamber is not at or near the distal region. In an embodiment, the fluid outlet of the fluid collection assembly may be at or near the gravimetric low point of the chamber and spaced from the distal region. In an embodiment, as illustrated in FIGS. 3A and 3B, the conduit may extend from the fluid outlet that is spaced from the expected gravimetric low point to a location in the chamber that is at or near the expected gravimetric low point.

FIG. 3A is a cross-sectional schematic of a fluid collection assembly 300, according to an embodiment. FIG. 3B is a cross-sectional schematic of the fluid collection assembly 300 taken along plane 3B-3B shown in FIG. 3A, according to an embodiment. Except as otherwise disclosed herein, the fluid collection assembly 300 may be the same or substantially similar to any of the fluid collection assemblies disclosed herein. For example, the fluid collection assembly 300 may include distal region 304, a proximal end region 306, at least one intermediate portion 308 extending from the distal region 304 to the proximal end region 306, and at least one LAF 316. The fluid collection assembly 300 may also include a fluid impermeable barrier 302 defining at least a chamber 310 and a fluid outlet 314. The fluid collection assembly 300 may further includes at least one porous material 320 disposed in the chamber 310.

In an embodiment, the gravimetric low point of the chamber 310 is expected to be at a location that is spaced from the fluid outlet 314. For example, the fluid outlet 314 may be located at or near the distal region 304 of the fluid collection assembly 300 while the gravimetric low point of the chamber 310 is expected to be at or near the proximal end region 306. It is noted that the fluid outlet 314 may be located at a location other than the distal region 304 (e.g., the intermediate portion 308 and/or near the proximal end region 306). It is also noted that the gravimetric low point of the chamber 310 may be at a location other than near the proximal end region 306 depending on at least one of the position of the patient (e.g., standing, sitting, or lying down) and/or the direction that the penis extends when the penis is erect or flaccid.

The conduit 342 may extend from the fluid outlet 314 to the expected gravimetric low point of the chamber 310 such that the inlet 354 of the conduit 342 is at or near the expected gravimetric low point. For example, in the illustrated embodiment, the conduit 342 may extend from the fluid outlet 314, at least one of between the fluid impermeable barrier 302 and the porous material 320, through the porous material 320 (e.g., through a bore formed in the porous material 320), or in an unoccupied spaced defined partially by the porous material 320 that also receives the penis (not shown). Positioning the inlet 354 of the conduit 342 at or near the expected gravimetric low point of the chamber 310 may facilitate removal of the bodily fluids from the chamber 310 since at least some of the bodily fluids may flow towards the gravimetric low point of the chamber 310.

In an embodiment, the chamber 310 may include a substantially unoccupied space at or near the proximal end region 306 or any other location that may be expected to be the gravimetric low point of the chamber 310. The substantially unoccupied space may be a reservoir 352 that is configured to temporarily store the bodily fluids therein.

In an embodiment, the conduit of any of the fluid collection assemblies disclosed herein may define a plurality of inlets thereby allowing the conduit to receive bodily fluids from a variety of locations in the chamber. In an embodiment, the conduit of any of the fluid collection assemblies disclosed herein may include a plurality of conduits and an inlet of at least one of the plurality of conduits is positioned in a location that is different than an inlet of at least one other one of the plurality of conduits thereby allowing the conduits to receive bodily fluids from a variety of locations in the chamber.

The fluid collection assemblies illustrated in FIGS. 1A-3B are illustrated as including only a single intermediate portion. However, it is noted that the fluid collection assemblies disclosed herein may include any suitable number of intermediate portions, such as 2 intermediate portions, 3 intermediate portions, 4 intermediate portions, 5 intermediate portions, or 6 or more intermediate portions. Each of the plurality of intermediate portions may be separated by an LAF (e.g., slit, perforations, etc.).

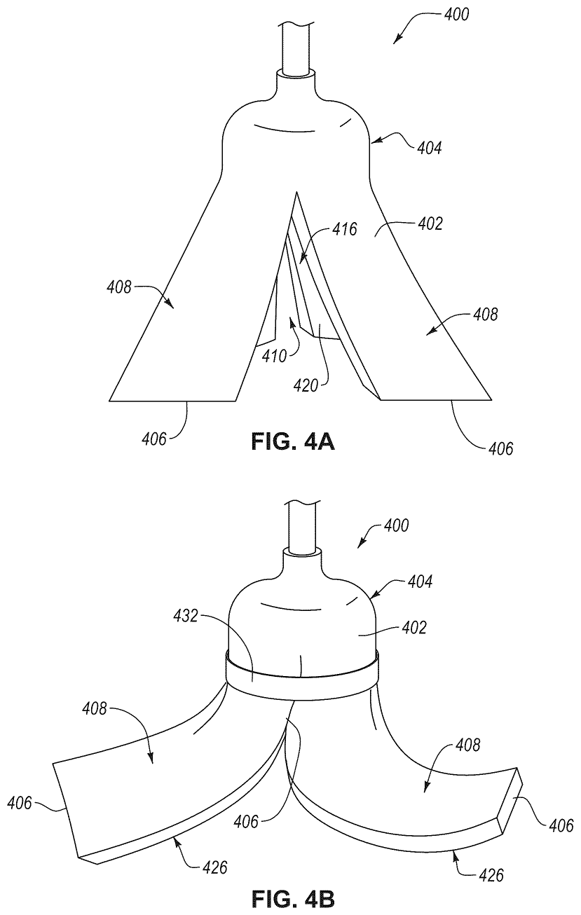

FIG. 4A is an isometric view of a fluid collection assembly 400 that includes a two intermediate portions 408, according to an embodiment. Except as otherwise disclosed herein, the fluid collection assembly 400 is the same or substantially similar to any of the fluid collection assemblies disclosed herein. For example, the fluid collection assembly 400 may include a distal region 404 and proximal end regions 406 spaced from the distal region 404. The fluid collection assembly 400 also includes a fluid impermeable barrier 402 defining at least a chamber 410 and at least one porous material 420 disposed in the chamber 410.

The fluid collection assembly 400 includes two intermediate portions 408 each extending from the distal region 404 to the proximal end regions 406. The two intermediate portions 408 are separated from each other by two LAFs 416. In the illustrated embodiment, the two intermediate portions 408 may be generally aligned linearly and/or may extend generally parallel to each other as the two intermediate portions 408 extend from the distal region 404. However, the two intermediate portions 408 may also extend from the distal region 404 while not being at least one of generally aligned linearly with each other or generally parallel to each other.

The intermediate portions 408 may be the same or substantially similar to any of the intermediate portions disclosed herein. For example, the intermediate portions 408 may include a fluid impermeable barrier 402 and at least one porous material 420 disposed adjacent to at least a portion of the fluid impermeable barrier 402 of each of the intermediate portions 408. The intermediate portions 408 may also exhibit any shape disclosed herein. For example, the intermediate portions 408 may exhibit a generally rectangular shape, a generally semi-cylindrical shape, a generally parallelepiped shape, or any other suitable shape.

In an embodiment, the two intermediate portions 408 are substantially the same. For example, the two intermediate portions 408 may exhibit the same size, the same shape, etc. In an embodiment, the two intermediate portions 408 may be different from each other in one or more aspects. For example, the intermediate portions 408 may exhibit different sizes, different shapes, one of the intermediate portions 408 may include a porous material 420 that is different than a porous material 420 of the other intermediate portion 408, one intermediate portion 408 may include a reservoir and/or a conduit extending therethrough while the other intermediate portion 408 does not, one intermediate portion 408 may include the fluid outlet 414, etc.

FIG. 4B is an isometric view of the fluid collection assembly 400 during use, according to an embodiment. The penis (patient not shown for clarity) may be disposed in the chamber 410 such that the penis is adjacent or at least proximate to the distal region 404. At least a portion of intermediate portions 408 may be flared (as shown) or folded to reduce the length of the fluid collection assembly 400 such that the penis is within, adjacent or at least proximate to the distal region 404. The fluid collection assembly 400 may include a retainer 432 to prevent or at least inhibit the unflared portions of the intermediate portions 408 from flaring.

In an embodiment, the flared portions 426 of the intermediate portions 408 may be flared outwardly until each of the flared portions 426 exhibits a shape that generally corresponds to the shape of the region about the penis that the flared portions 426 contact. The portions of the body that the flared portions 426 correspond to may depend on the orientation (e.g., rotation) of the fluid collection assembly 400 exhibit relative to the patient. In an example, the flared portions 426 may be flared until at least a portion of the flared portions 426 exhibit a shape that generally corresponds to the shape of the thighs of the patient. In an example, the flared portions 426 may be flared until at least a portion of the flared portion 426 of one of the intermediate portions 408 exhibit a shape that generally corresponds to the shape of at least a portion of the testicles of the patient while the flared portion 426 of the other intermediate portions 408 exhibits a shape that generally corresponds to the shape of the mons pubis. In an embodiment, the flared portions 426 may be flared such that the flared portions 426 do not exhibit a shape that corresponds to the shape of the region about the penis. For example, the flared portions 426 may be folded upwardly such that at least a portion of the fluid impermeable barrier 402 of the flared portions 426 is adjacent to the fluid impermeable barrier 402 of at least a portion of the rest of the fluid collection assembly 400.

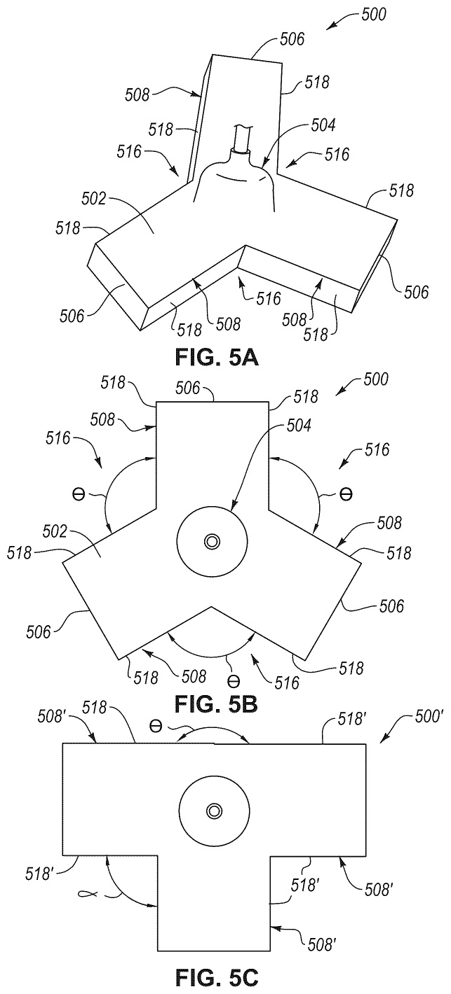

As previously discussed, the fluid collection assembly 400 includes two intermediate portions. However, any of the fluid collection assemblies disclosed herein may include more than two intermediate portions, such as three intermediate portions as illustrated in FIGS. 5A-5C. FIG. 5A is an isometric view of a fluid collection assembly 500, according to an embodiment. FIG. 5B is a top plan view of the fluid collection assembly 500, according to an embodiment. Except as otherwise disclosed herein, the fluid collection assembly 500 may be the same or substantially similar to any of the fluid collection assemblies disclosed herein. For example, the fluid collection assembly 500 may include a distal region 504, proximal end regions 506 spaced from the distal region 504, three intermediate portions 508, and at least one LAF 516 disposed between each of the three intermediate portions 508. For illustrative purposes, the intermediate portions 508 are illustrated as being flared though, it is noted, the intermediate portions 508 may not be flared. The fluid collection assembly 500 also includes a fluid impermeable barrier 502 and at least one porous material (not shown).

As previously discussed, the fluid collection assembly 500 includes three intermediate portions 508. The three intermediate portions 508 allows the fluid collection assembly 500 to be attached to more regions of the body of the patient that is using the fluid collection assembly 500 than if the fluid collection assembly 500 only included one or two intermediate portions. In an example, the three intermediate portions 308 allows the fluid collection assembly 500 to be attached to the two thighs and the mons pubis of the patient. Allowing the fluid collection assembly 500 to be attached to more portions of the bodily causes the fluid collection assembly 500 to pull less on each portion of the patient thereby increasing patient comfort In an example, the three intermediate portions 508 allows two of the intermediate portions 508 to be attached above the penis (i.e., in a region generally between the penis and head of the patient) or to the side of the penis while the remaining intermediate portion 508 may be attached below the penis (i.e., in a region generally between the penis and the feet of the patient). In an example, the three intermediate portions 508 allows two of the intermediate portions 508 to be attached below or to the sides of the penis while the remaining intermediate portion 508 may be attached above the penis. In an embodiment, the three intermediate portions 508 may minimize pivoting and increase the likelihood that the fluid collection assembly 500 stays attached to the patient than if the fluid collection assembly 500 included fewer intermediate portions.

The three intermediate portions 508 may allow the fluid collection assembly 500 to be attached to the patient without attaching one or more of the intermediate portions 508 to sensitive portions of the patient (e.g., testicles) compared to a fluid collection assembly that includes two or fewer intermediate portions. In an example, the intermediate portions 508 may be attached to the mons pubis without also attaching the intermediate portions 508 to the testicles whereas attaching one of the intermediate portions 408 of the fluid collection assembly 400 illustrated in FIG. 4A to the mons pubis may also cause the other intermediate portion to be attached to the testicles. In an example, the fluid collection assembly 500 may be positioned on the patient such that one of the intermediate portions 508 may be attached to the testicles.

It is noted that generally, increasing the number of intermediate portions increases the comfort of using the fluid collection assembly by increasing the number of locations to which the intermediate portions may be attached while also avoiding more sensitive portions of the patient.

Referring to FIG. 5B, the intermediate portions 508 may be “equidistantly spaced” when each angle θ between two adjacent lateral edges 518 of different intermediate portions 508 are the same or substantially the same. In the illustrated embodiment, the angle θ is about 120° when the three intermediate portions 508 are equidistantly spaced. Equidistantly spacing intermediate portions 508 may allow each of the intermediate portions 508 to more equally carry the weight of the fluid collection assembly 500 when the intermediate portions 508 are attached to the patient as the patient moves. It is noted that the other fluid collection assemblies disclosed herein may have their intermediate portions equidistantly spaced. In an example, the fluid collection assembly 400 illustrated in FIGS. 4A and 4B may have equidistantly spaced intermediate portions 408 when the angle between the two intermediate portions is about 180° (i.e., aligned linearly). In an example, a fluid collection assembly with four equidistantly spaced intermediate portions has an angle between adjacent set of intermediate portions that is about 90°, a fluid collection assembly with five equidistantly spaced intermediate portions has an angle between adjacent set of intermediate portions that is about 72°, and so forth.

The intermediate portions of any of the fluid collection assemblies disclosed herein may have non-equidistantly spaced intermediate portions (e.g., an angle between two adjacent lateral edges of a first set of two different intermediate portions is different than an angle between two other adjacent lateral edges of a second set of two different intermediate portions). For example, FIG. 5C is a top plan view of a fluid collection assembly 500′ that includes a plurality of intermediate portions 508′ that are not equidistantly spaced, according to an embodiment. The fluid collection assembly 500′ is similar to any of the fluid collection assemblies disclosed herein (e.g., the fluid collection assembly 500 illustrated in FIGS. 5A and 5B) in one or more aspects. For example, the fluid collection assembly 500′ includes a plurality (e.g., three) intermediate portions 508′. However, unlike the other fluid collection assemblies disclosed herein, the intermediate portions 508′ are not equidistantly spaced. For example, an angle θ is measure between adjacent lateral edges 518′ of two different intermediate portions 508′ and an angle α is measured between other adjacent lateral edges 518′ of two different intermediate portions 508′, where the angle θ and the angle α are different. The angles between the other adjacent set of intermediate portion(s) may be the same or different than at least one of the angle θ and the angle α.

The angle θ and the angle α may be selected such that the intermediate portions 508′ are attached to selected portions of the body and/or avoid certain portions of the body. For example, the angle θ may be selected such that the intermediate portions 508′ that the angle θ is measured between avoids the testicles. For instance, the angle θ may be selected to be about 120° to about 200° (e.g., about) 180° thereby allowing the corresponding set of adjacent intermediate portions 508′ are attached to the thighs instead of the testicles. The angle α may be selected such that one of the intermediate portions 508′ that the angle α is measured between contacts the mons pubis or another location other than the testicles. As such, the angle θ and the angle a are selected to avoid attaching or contacting one of the intermediate portions 508′ against the testicles.

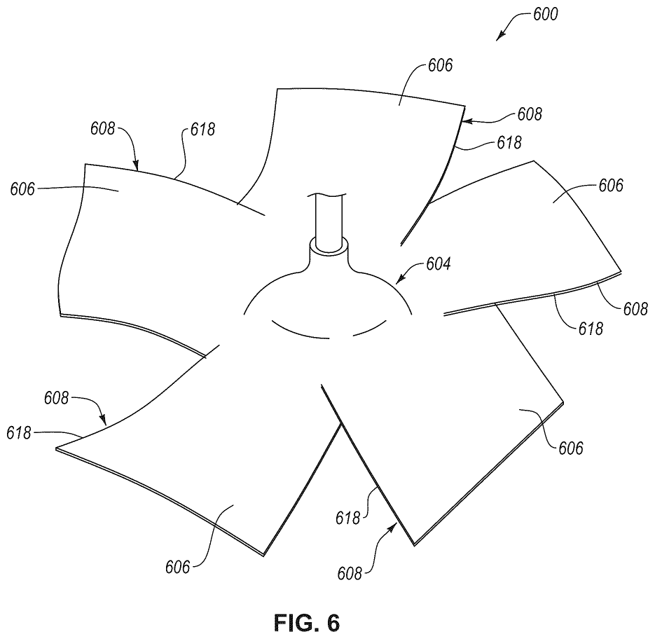

In some embodiments, the intermediate portions of any of the fluid collection assemblies disclosed herein may overlap. For example, FIG. 6 is an isometric view of a fluid collection assembly 600 that includes overlapping intermediate portions 608, according to an embodiment. Except as otherwise disclosed herein, the fluid collection assembly 600 is the same of substantially similar to any of the fluid collection assemblies disclosed herein. For example, the fluid collection assembly 600 includes a distal region 604, proximal end regions 606 spaced from the distal region 604, and a plurality of intermediate portions 608 extending from the distal region 604 to the proximal end regions 606.

As previously discussed, the fluid collection assembly 600 includes a plurality of intermediate portions 608 and at least a portion of at least some of the plurality of intermediate portions 608 overlap with each other. The intermediate portions 608 overlap when one intermediate portion 608 covers a portion of another intermediate portion 608 (e.g., an adjacent intermediate portion 608) when the intermediate portions 608 are completely flared. The overlapping intermediate portion 608 generally covers a portion of the adjacent intermediate portion 608 that is adjacent to the distal region 604.

The overlapping of the intermediate portions 608 prevents or at least inhibits bodily fluids from leaking from the chamber (not shown) of the fluid collection assembly 600. For example, one of the more likely locations of the fluid collection assembly 600 that the bodily fluids may leak from is through the LAF adjacent to the distal region 604. In fluid collection assemblies that do not include overlapping intermediate portions, any bodily fluids that flow around the lateral edge of an intermediate portion adjacent to the distal region may be able to leak from the such a fluid collection assembly. However, in the fluid collection assembly 600 illustrated in FIG. 6, the bodily fluids that flow around a lateral edge 618 of a first intermediate portion 608 that is adjacent to the distal region 604 must flow between the first intermediate portion 608 and the intermediate portion 608 that overlaps the first intermediate portion 608 without being received by the porous material before the bodily fluids may leak from the fluid collection assembly 600. As such, the overlapping intermediate portions 608 decrease the likelihood that the bodily fluids leak from the fluid collection assembly 600 than if the intermediate portions 608 did not overlap.

In an embodiment, all of the intermediate portions 608 are partially overlapped by another intermediate portion 608 and, by extension, all of the intermediate portions 608 partially overlap another intermediate portion 608. In an embodiment, at least one of the intermediate portions 608 is overlapped by two intermediate portions adjacent intermediate portion 608. In an embodiment, at least one of the intermediate portion 608 is not overlapped and/or does not overlap another intermediate portion 608.

The fluid collection assemblies illustrated in FIGS. 1A-6 are illustrated as having 1, 2, 3, or 5 intermediate portions. However, the fluid collection assemblies disclosed herein may include any number of intermediate portions. For example, the fluid collection assemblies disclosed herein may include 4 or 6 or more intermediate portions. It is noted that it is generally believed that increasing the number of intermediate portions beyond 6 may adversely affect the operation of the fluid collection assembly. For instance, increasing the number of intermediate portions of a fluid collection assembly beyond 6 may make attaching the fluid collection assembly patient without attaching the intermediate portions to themselves or to the wrong portion of the patient difficult. Further, increasing the number of intermediate portions of the fluid collection assembly beyond 6 may increase the chances of bodily fluids leaking from the fluid collection assembly due to the number of LAFs through which the bodily fluids may leak.

The fluid collection assemblies illustrated in FIGS. 4A-6 include intermediate portions that extend a substantially equal distance from the distal regions thereof. However, the fluid collection assemblies disclosed herein may include intermediate portions that do not extend a substantially equal distance from the distal regions thereof, as shown in FIGS. 7A-7B. The fluid collection assembly that includes intermediate portions that do not extend a substantially equal distance from the distal regions thereof may allow such a fluid collection assembly to be positioned against the penis that would be difficult to do if the intermediate portions extended a substantially equal distance from the distal region. FIG. 7A is an isometric view of a fluid collection assembly 700, according to an embodiment. Except as otherwise disclosed herein, the fluid collection assembly 700 is the same or substantially similar to any of the fluid collection assemblies disclosed herein. For example, the fluid collection assembly 700 includes a distal region 704, proximal end regions 706 spaced from the distal region 704, and a plurality of intermediate portions extending from the distal region 704 to the proximal end region 706.

The fluid collection assembly 700 includes a first intermediate portion 708a and a second intermediate portion 708b. The first intermediate portion 708a extends a first distance from the distal region 704 and the second intermediate portion 708b extends a second distance from the distal region 704, wherein the second distance is greater than the first distance. In other words, the first intermediate portion 708a exhibits a length that is less than a length of the second intermediate portion 708b. As will be discussed in more detail with regards to FIG. 7B, the different lengths of the first and second intermediate portion 708a, 708b may facilitate attachment of the fluid collection assembly 700 to a penis 724 (shown in FIG. 7B).

The proximal end region 706 formed by the first intermediate portion 708a or a portion of the first intermediate portion 708a adjacent to the proximal end region 706 is configured to abut or at least partially surround the penis 724 (shown in FIG. 7B). For example, some of the bodily fluids discharged from the penis 724 into the chamber 710 (shown in FIG. 7B) may flow down the shaft 757 of the penis 724 or the penis may lay on either side rather than straight up. The portion(s) of the first intermediate portion 708a that abut or at least partially surround the penis 724 may receive or at least maintain in the chamber 710 at least some of the bodily fluids that flow down the shaft 757 of the penis 724 or receive bodily fluids that otherwise remain on the shaft. Thus, the portion(s) of the first intermediate portion 708a that abut or at least partially surround the penis 724 may minimize leakage of bodily fluids from the chamber 710.

In an embodiment, the first intermediate portion 708a may define a cutout 756. The cutout 756 may extend inwardly from (as shown) from the proximal end region 706 or may be a hole proximate the proximal end region 706 that is completely surrounded by the first intermediate portion 708. The cutout 756 is configured to receive the penis 724 such that the first intermediate portion 708a partially surrounds (when the cutout 756 extends inwardly from the proximal end region 706) or completely surrounds (when the cutout 756 is a hole) the penis 724. The cutout 756 allows the first intermediate portion 708a to be adjacent to or abut more of the penis 724 than if the first intermediate portion 708a did not include the cutout 756. As such, the cutout 756 allows the first intermediate portion 708a to receive or at least maintain in the chamber 710 more of the bodily fluids that if the first intermediate portion 708a did not include the cutout 756.

FIG. 7B is a cross-sectional schematic of the fluid collection assembly 700 being used with a penis 724, according to an embodiment. As shown, the first intermediate portion 708a is positioned adjacent to or abuts the penis 724, such as the shaft 757 or the base 758 of the penis 724. When the first intermediate portion 708a includes a cutout 756, the penis 724 may be at least partially positioned in the cutout 756. The urethral opening (e.g., glans 760) of the penis 724 is positioned within or adjacent to the distal region 704 such that bodily fluids discharged from the urethral opening may be preferentially received by the distal region 704 and removed from the chamber 710 using the fluid outlet 714. A portion of the second intermediate portion 708b may then be positioned adjacent to an opposing side of the shaft 757 of the penis 724 than the first intermediate portion 708a. The portion of the second intermediate portion 708b that is not adjacent to the shaft 757 of the penis 724 may be positioned against the mons pubis 728 of the patient. As illustrated in FIG. 7B, the second intermediate portion 708b may travel a longer distance from the distal region 704 than the first intermediate portion 708a and, for this reason, the second intermediate portion 708b may exhibit a length that is greater than the first intermediate portion 708a. A portion of the first intermediate portion 708a (e.g., edges of the first intermediate portion 708a) and a portion of the second intermediate portion 708b (e.g., edges of the second intermediate portion 708b) may be attached together or at least positioned adjacent to each other to minimize bodily fluids leaking between the first and second intermediate portions 708a, 708b.

The fluid collection assemblies illustrated in FIGS. 1A-7B are illustrated as including a distal region that is a sump. However, in some embodiments, the fluid collection assemblies disclosed herein may include a distal region that is not a sump. For example, FIG. 8 is an isometric view of a fluid collection assembly 800 that includes a distal region 804 that is not a sump, according to an embodiment. Except as otherwise disclosed herein, the fluid collection assembly 800 is the same or substantially similar to any of the fluid collection assemblies disclosed herein. For example, the fluid collection assembly 800 may include the distal region 804, at least one proximal end region 806, and at least one intermediate portion 808 extending from the distal region 804 to the proximal end region 806.

As previously discussed, the distal region 804 of the fluid collection assembly 800 is not a sump. Such a distal region 804 may allow the fluid collection assembly 800 to lie flat on a surface or otherwise conform to the shape of a surface. The ability of the fluid collection assembly 800 to lie flat on a surface or otherwise conform to the shape of the surface may allow the fluid collection assembly 800 to be more effectively used with a buried penis. For example, the distal region 804 of the fluid collection assembly 800 may lie flat across the buried penis thereby allowing the urethral opening to be positioned closer to the fluid outlet 814 than if the distal region 804 included a sump. It is noted that a fluid collection assembly may still be used with a buried penis if the distal region thereof is a sump but that the sump may increase the distance between the urethral opening of the buried penis and the fluid outlet.

The at least one intermediate portion 808 of the fluid collection assembly 800 allows the fluid collection assembly 800 to be used with non-buried penises. For example, at least a portion of the lateral edges 818 may be positioned together thereby allowing the intermediate portion 808 in conjunction with the distal region 804 to form a sump-like shape. The non-buried penis may be disposed in the sump-like shape.

It is noted that the fluid outlet 814 may extend outwardly from the rest of the fluid collection assembly 800 when the fluid collection assembly 800 lies flat. Extending the fluid outlet 814 outwardly from the rest of the fluid collection assembly 800 may facilitate attaching the conduit 842 thereto. However, the fluid outlet 814 is too small to form a sump in which the glans of the penis may be positioned therein. Thus, the fluid collection assembly 800 may include a distal region 804 that is not a sump but also include a fluid outlet 814 extending outwardly from the rest of the fluid collection assembly 800.

FIG. 9 is a block diagram of a fluid collection system 901 for fluid collection, according to an embodiment. The fluid collection system 901 includes a fluid collection assembly 900 that is similar or identical to any of the fluid collection assemblies disclosed herein in one or more aspects. The fluid collection system 901 also includes a fluid storage container 907, and a vacuum source 909. The fluid collection assembly 900, the fluid storage container 907, and the vacuum source 909 may be fluidly coupled to each other via one or more conduits 942. For example, fluid collection assembly 900 may be operably coupled to one or more of the fluid storage container 907 or the vacuum source 909 via the conduit 942. Bodily fluids (e.g., urine or other bodily fluids) collected in the fluid collection assembly 900 may be removed from the fluid collection assembly 900 via the conduit 942 which protrudes into the fluid collection assembly 900. For example, an inlet of the conduit 942 may extend into the fluid collection assembly 900, such as to a reservoir therein. The outlet of the conduit 942 may extend into the fluid storage container 907 or the vacuum source 909. Suction force may be introduced into the chamber of the fluid collection assembly 900 via the inlet of the conduit 942 responsive to suction (e.g., vacuum) force applied at the outlet of the conduit 942.

The suction force may be applied to the outlet of the conduit 942 by the vacuum source 909 either directly or indirectly. The suction force may be applied indirectly via the fluid storage container 907. For example, the outlet of the conduit 942 may be disposed within the fluid storage container 907 and an additional conduit 942 may extend from the fluid storage container 907 to the vacuum source 909. Accordingly, the vacuum source 909 may apply suction to the fluid collection assembly 900 via the fluid storage container 907. The suction force may be applied directly via the vacuum source 909. For example, the outlet of the conduit 942 may be disposed within the vacuum source 909. An additional conduit 942 may extend from the vacuum source 909 to a point outside of the fluid collection assembly 900, such as to the fluid storage container 907. In such examples, the vacuum source 909 may be disposed between the fluid collection assembly 900 and the fluid storage container 907.

The fluid storage container 907 is sized and shaped to retain the bodily fluids therein. The fluid storage container 907 may include a bag (e.g., drainage bag), a bottle or cup (e.g., collection jar), or any other enclosed container for storing bodily fluids such as urine. In some examples, the conduit 942 may extend from the fluid collection assembly 900 and attach to the fluid storage container 907 at a first point therein. An additional conduit 942 may attach to the fluid storage container 907 at a second point thereon and may extend and attach to the vacuum source 909. Accordingly, a vacuum (e.g., suction) may be drawn through fluid collection assembly 900 via the fluid storage container 907. Fluid, such as urine, may be drained from the fluid collection assembly 900 using the vacuum source 909.

The vacuum source 909 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 909 may provide a vacuum or suction to remove fluid from the fluid collection assembly 900. In some examples, the vacuum source 909 may be powered by one or more of a power cord (e.g., connected to a power socket), one or more batteries, or even manual power (e.g., a hand operated vacuum pump). In some examples, the vacuum source 909 may be sized and shaped to fit outside of, on, or within the fluid collection assembly 900. For example, the vacuum source 909 may include one or more miniaturized pumps or one or more micro pumps. The vacuum sources 909 disclosed herein may include one or more of a switch, a button, a plug, a remote, or any other device suitable to activate the vacuum source 909.

The fluid collection assemblies disclosed herein are discussed as being used with a penis. For example, the fluid collection assemblies are disclosed as being positioned on, around, or above a penis. However, it is noted that the fluid collection assemblies disclosed herein may collect bodily fluids from anatomy other than a penis. In an embodiment, the fluid collection assemblies disclosed herein may be positioned on, around, or above a female urethral opening (e.g., vaginal) thereby allowing the fluid collection assemblies to remove urine, blood, vagina discharge, or other relevant bodily fluids from the vagina. In an embodiment, the fluid collection assemblies disclosed herein may be positioned on, around, or above a wound thereby allowing the fluid collection assemblies to receive and remove blood, puss, serous fluid, or other relevant bodily fluids from the wound. In an embodiment, the fluid collection assemblies may be disposed on any other anatomical feature of a patient to remove moisture, oil, or other bodily fluids from the anatomical feature, such as a finger of a surgeon.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more perforations extending therefrom, is oblong, is the same as the disclosed shape, etc.

What is claimed is:

1. A fluid collection assembly, comprising:
a distal region;
at least one proximal end region spaced from the distal region;
a plurality of intermediate portions extending from the at least one proximal end region to the distal end region, the plurality of intermediate portions configured to be lie substantially flat when completely flared;
a plurality of length adjusting features extending from the at least one proximal end region towards the distal region, the plurality of length adjusting features configured to allow a length of the fluid collection assembly to change, the plurality of length adjusting features separating the plurality of intermediate portions from each other;
a fluid impermeable barrier forming at least a portion of the distal region, the at least one proximal end region, and the plurality of intermediate portions, the fluid impermeable barrier at least defining a chamber, an opening at the proximal end region, and a fluid outlet; and
at least one porous material disposed in the chamber, the at least one porous material configured to receive bodily fluids and move the bodily fluids towards the fluid outlet.

2. The fluid collection assembly of claim 1, wherein the distal region is a sump.

3. The fluid collection assembly of claim 2, wherein the distal region forms a bulge when the plurality of intermediate portions are completely flared.

4. The fluid collection assembly of claim 1, wherein the plurality of intermediate portions includes a first intermediate portion and a second intermediate portion, and wherein the first intermediate portion exhibits a first length and the second intermediate portion exhibits a second length, the first length and the second length measured from the distal region, the first length less than the second length.

5. The fluid collection assembly of claim 4, wherein the plurality of intermediate portions only include the first intermediate portion and the second intermediate portion.

6. The fluid collection assembly of claim 5, wherein the first intermediate portion defines a cutout that is configured to receive at least a portion of a penis, the cutout extending completely through a thickness of the first intermediate portion.

7. The fluid collection assembly of claim 6, wherein the cutout extending from the at least one proximal end region of the first intermediate portion partially towards the distal region.

8. The fluid collection assembly of claim 1, wherein one of the plurality of intermediate portions defines a cutout that is configured to receive at least a portion of a penis, the cutout extending completely through a thickness of the one of the plurality of intermediate portions.

9. The fluid collection assembly of claim 1, wherein the at least one porous material includes a plurality of porous material slits adjacent, one of the plurality of porous material slits adjacent to each of the plurality of length adjusting features.

10. The fluid collection assembly of claim 9, wherein:
the at least one porous material including one or more edges adjacent to the proximal end region and adjacent to each of the plurality of porous material slits; and
the fluid impermeable barrier includes a plurality of inwardly extending portions extending distally into the chamber from an exterior portion of the fluid impermeable barrier, the plurality of inwardly extending portions at least partially covering the one or more edges adjacent to the plurality of porous material slits.

11. The fluid collection assembly of claim 10, wherein the plurality of inwardly extending portions at least partially covering the one or more edges adjacent to the proximal end region.

12. The fluid collection assembly of claim 10, wherein the fluid impermeable barrier including at least one chamber extending portion extending from at least one the plurality of inwardly extending portions, the at least one inwardly extending portion covering a portion of at least one interior surface of the at least one porous material.

13. The fluid collection assembly of claim 1, wherein the at least one porous material extends into and across the sump.

14. The fluid collection assembly of claim 1, wherein the at least one porous material extends across an inlet of the fluid outlet.

15. The fluid collection assembly of claim 1, wherein the plurality of length adjusting features including a slit.

16. The fluid collection assembly of claim 1, wherein the plurality of length adjusting features including a plurality of perforations.

17. A fluid collection system, comprising:
a fluid collection assembly including:
a distal region;
at least one proximal end region spaced from the distal region;
a plurality of intermediate portions extending from the at least one proximal end region to the distal end region, the plurality of intermediate portions configured to be lie substantially flat when completely flared;
a plurality of length adjusting features extending from the at least one proximal end region towards the distal region, the plurality of length adjusting features configured to allow a length of the fluid collection assembly to change, the plurality of length adjusting features separating the plurality of intermediate portions from each other;
a fluid impermeable barrier forming at least a portion of the distal region, the at least one proximal end region, and the plurality of intermediate portions, the fluid impermeable barrier at least defining a chamber, an opening at the proximal end region, and a fluid outlet; and
at least one porous material disposed in the chamber, the at least one porous material configured to receive bodily fluids and move the bodily fluids towards the fluid outlet;
a fluid storage container configured to hold one or more bodily fluids;
a vacuum source fluidly coupled to one or more of the fluid storage container or the fluid collection assembly via a conduit, the vacuum source configured to draw fluid from the fluid collection assembly via the conduit.

18. A method of using a fluid collection assembly, the method comprising:
providing the fluid collection assembly, the fluid collection assembly including:
a distal region;
at least one proximal end region spaced from the distal region;
a plurality of intermediate portions extending from the at least one proximal end region to the distal end region, the plurality of intermediate portions configured to be lie substantially flat when completely flared;
a plurality of length adjusting features extending from the at least one proximal end region towards the distal region, the plurality of length adjusting features configured to allow a length of the fluid collection assembly to change, the plurality of length adjusting features separating the plurality of intermediate portions from each other;
a fluid impermeable barrier forming at least a portion of the distal region, the at least one proximal end region, and the plurality of intermediate portions, the fluid impermeable barrier at least defining a chamber, an opening at the proximal end region, and a fluid outlet; and
at least one porous material disposed in the chamber, the at least one porous material configured to receive bodily fluids and move the bodily fluids towards the fluid outlet;
partially flaring at least one of the plurality of intermediate portions; and
positioning a penis of an individual in the chamber.

19. The method of claim 18, wherein positioning a penis of an individual in the chamber includes positioning the penis in a cutout defined by one of the plurality of intermediate portions, the cutout extending completely through a thickness of the one of the plurality of intermediate portions.

20. The method of claim 19, wherein positioning the penis in a cutout includes positioning the one of the plurality of intermediate portions adjacent to one side of the penis; and
further comprising positioning another one of the plurality of intermediate portions adjacent to an opposing side of the penis.
